(19) United States
(12) Patent Application Publication
(10) Pub. No.: US 2004/0063158 A1
(43) Pub. Date: Apr. 1, 2004

(54) Method of determining surface binding capacity

(76) Inventors: Ivan Alferiev, Clementon, NJ (US); Ilia Fishbein, Philadelphia, PA (US); Robert J. Levy, Merion Station, PA (US)

Correspondence Address: CAESAR, RIVISE, BERNSTEIN,, COHEN & POKOTILOW, LTD., 12TH FLOOR, SEVEN PENN CENTER, 1635 MARKET STREET, PHILADELPHIA, PA, 19103-2212, US

(21) Appl. No.: 10/672,225

(22) Filed: Sep. 26, 2003

Related U.S. Application Data
(60) Provisional application No. 60/413,460, filed on Sep. 26, 2002.

(51) Int. Cl. G01N 33/53; C07K 14/47

(52) U.S. Cl. 435/7.1; 530/404; 530/405

(57) ABSTRACT

A method of determining a binding capacity of a surface, the method including providing the surface containing a reactive moiety; providing a fluorophore including a fluorescent moiety adapted to emit a detectable signal; reacting the fluorophore with the reactive moiety to form a linking bond between the fluorophore and the reactive moiety; cleaving a cleavable bond to liberate the fluorescent moiety; and detecting the detectable signal to determine the binding capacity of the surface.

### BACKGROUND OF THE INVENTION

1. Field of Invention

This invention relates to the field of quantitatively measuring binding capacity of a surface containing reactive moieties.

2. Description of Related Art

It is known to modify polyurethanes with reactive moieties so that modified surfaces can react with molecules of interest, for example, bioactive molecules. U.S. Pat. No. 6,320,011 to Levy et al. discloses polyurethane (PU) derivatized to contain pending geminal bisphosphonate groups. Derivatized PU can then react with proteins, cells, antibodies, and/or enzymes.

Prior art polyurethanes that are suitably modified for the covalent immobilization of various bioactive molecules are rather limited in number and utility. For example, polyurethanes containing pendant carboxy groups were synthesized in order to covalently attach recombinant hirudin (Phaneuff, M. D. et al. “Covalent Linkage of Recombinant Hirudin to a Novel ionic Poly(carbonate)urethane Polymer With Protein Binding Sites: Determination of Surface Antithrombin Activity,” Artif. Organs 1998; 22:657-65). Alternatively, polyurethanes with pendant epoxy groups have been used for the covalent immobilization of collagen (Huang L. L. H. et al. “Comparison of Epoxides on Grafting Collagen to Polyurethane and Their Effects on Cellular Growth,” J. Biomed. Mater. Res. 1998; 39:630-6).

It is known to use florophores to label biomolecules for detection purposes and for studying structures and interactions. Assays using flourophores are conducted in a solution, wherein concentrations of flourophores are diluted enough to avoid quenching.

Despite the foregoing developments, there is a need in a method, which provides an accurate and simple method of measuring a binding capacity of a surface by quantifying an amount of reactive moieties on a surface available for binding molecules.

All references cited herein are incorporated herein by reference in their entireties.

### BRIEF SUMMARY OF THE INVENTION

Accordingly, the invention provides a method of determining a binding capacity of a surface, the method comprising providing the surface containing a reactive moiety; providing a fluorophore comprising a fluorescent moiety adapted to emit a detectable signal; reacting the fluorophore with the reactive moiety to form a linking bond between the fluorophore and the reactive moiety; cleaving a cleavable bond to liberate the fluorescent moiety; and detecting the detectable signal to determine the binding capacity of the surface.

Also provided is a method of determining a binding capacity of a surface, the method comprising: providing the surface containing a reactive moiety; providing a fluorophore comprising a fluorescent moiety adapted to emit a detectable signal; reacting the fluorophore with the reactive moiety to form a linking bond between the fluorophore and the reactive moiety, wherein the linking bond is the cleavable bond and is a disulfide bond or an aromatic azo group; cleaving a cleavable bond to liberate the fluorescent moiety; and detecting the detectable signal to determine the binding capacity of the surface.

In certain embodiments, the cleavable bond is a disulfide bond.

In certain embodiments, the cleavable bond is the aromatic azo group represented by a formula:

—R²—N═N—

wherein R² is an aromatic compound selected from the group consisting of a heterocyclic group and an electron-deficient aromatic group.

In certain embodiments, the fluorophore is a thiol-containing fluorescent structure represented by a formula:

Fl—SH

wherein Fl is the fluorescent moiety and is a member selected from the group consisting of fluorescent L-cysteine, BODIPY-L-cysteine, fluorescein and derivatives thereof.

In certain embodiments, the thiol-containing fluorescent structure is a member selected from the group consisting of:

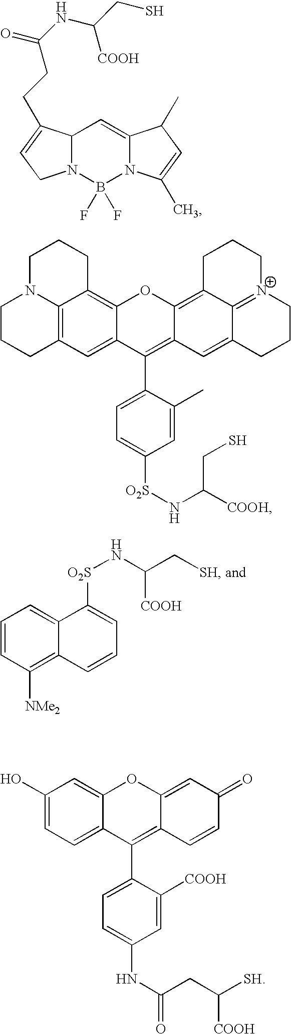

In certain embodiments, the fluorophore is a thiol-reactive fluorescent structure represented by a formula:

Fl—S—X

wherein X is a member selected from the group consisting of Cl, SO₃(C₁-C₆ alkyl), and S—R², wherein R² is a heterocyclic group or an electron-deficient aromatic group.

In certain embodiments, R is a pyridyl group or a phenyl group substituted with one or more electron-withdrawing substituents.

In certain embodiments, the thiol-reactive fluorescent structure is a member selected from the group consisting of:

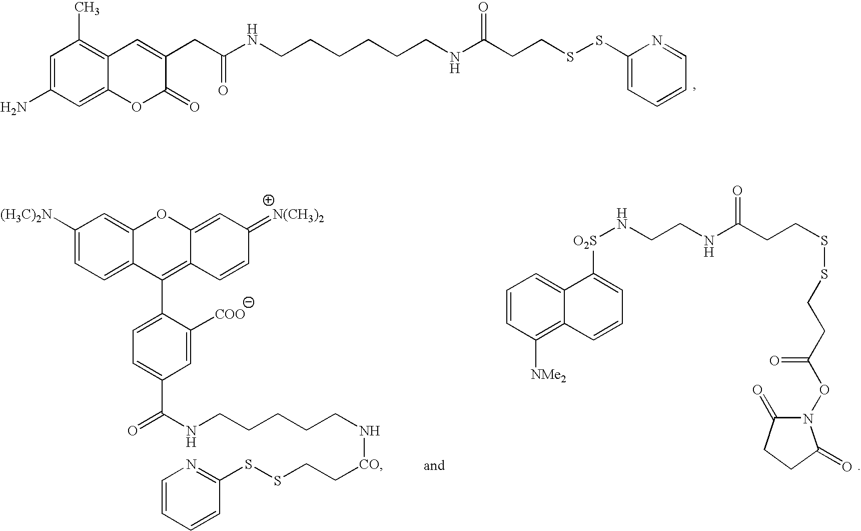

In certain embodiments, the fluorophore further comprises a functional group, wherein the functional group is bound to the fluorescent moiety by the cleavable bond and is reacted with the reactive moiety to form an uncleavable bond such that cleaving predominantly occurs at the cleavable bond.

In certain embodiments, the functional group is a member selected from the group consisting of an amino group, a thiol group, a protected thiol group, and an epoxy group.

In certain embodiments, the surface is a member selected from the group consisting of a polymer, a metal, a biomaterial, a ceramic, and a semiconductor. Preferably, the polymer is polyurethane.

In certain embodiments, the reactive moiety is a thiol, a thiol-reactive group or a group adapted to be converted into a thiol or a thiol-reactive group.

In certain embodiments, the reactive moiety is a thiol group or an amino group.

In certain embodiments, the reactive moiety is further reacted with 5,5′-dithio-bis(2-nitrobenzoic acid) or succinimidyl 3-(2-pyridyldithio)propionate.

In certain embodiments, the reactive moiety is a dithio group.

In certain embodiments, the cleavable bond is cleaved by using a reducing agent selected from the group consisting of dithiothreitol, β-mercaptoethanol, mercaptoethylamine hydrochloride, a borohydride, and a phosphine.

In certain embodiments, the borohydride is sodium borohydride.

In certain embodiments, the phosphine is a member selected from the group consisting of tris(2-cyanoethyl)phosphine, tris(2-carboxyethyl)phosphine and trimethylphosphine.

Further provided is a kit for practicing of the method of determining a binding capacity of a surface, the kit comprising a fluorophore.

In certain embodiments, the fluorophore comprises the fluorescent moiety and a linking bond precursor.

In certain embodiments, the linking bond precursor is adapted to form a cleavable disulfide bond or an aromatic azo group. In certain embodiments, the linking bond precursor is —SH. In certain embodiments, the linking bond precursor is represented by a formula:

—S—X

wherein X is a member selected from the group consisting of Cl, SO₃(C₁-C₆ alkyl), and S—R², wherein R² is a heterocyclic group or an electron-deficient aromatic group . . .

In certain embodiments, the fluorophore further comprises a functional group, wherein the functional group is bound to the fluorescent moiety by the cleavable bond and is adapted to react with the reactive moiety to form an uncleavable bond.

In certain embodiments, the functional group is a member selected from the group consisting of an amino group, a thiol group, a protected thiol group, and an epoxy group.

In certain embodiments, the uncleavable bond is an amide bond.

### BRIEF DESCRIPTION OF SEVERAL VIEWS OF THE DRAWINGS

The invention will be described in conjunction with the following drawings in which like reference numerals designate like elements and wherein:

FIG. 1 depicts a synthesis of a polyurethane having a pendant protected thiol group. A urethane amino nitrogen in a polyurethane schematically represented herein as 1 is bromoalkylated to obtain a bromobutyl derivative 2, in which the bromo substituent is subsequently substituted by thiolacetate to obtain polyurethane 3 having a pendant protected thiol group.

FIG. 2 shows a non-limiting example of a reaction sequence by which a quantitative assay of surface thiol-reactive groups is carried out. Polyurethane 3 is treated with the deprotecting reagent hydroxylamine (NH₂OH) to obtain polyurethane 4 having pendant thiol groups. The wavy lines represent R_{L}, an organic radical comprising at least one carbon atom. The thiol groups are then tagged with a fluorescent moiety by treating them sequentially with 5,5′-dithiobis(2-nitrobenzoic acid) (“DTNB”) and the thiol-containing fluorophore dansyl-L-cysteine (“Fl—SH”). The resultant polyurethane 6 comprises fluorescent moieites (“Fl”) attached to the polyurethane via disulfide bonds. Reduction of the disulfide bonds by tris(carboxyethyl)phosphine (“TCEP”) regenerates polyurethane 4 and liberates dansyl-L-cysteine (Fl—SH) into solution.

### DETAILED DESCRIPTION OF THE INVENTION

The invention was driven by the desire to develop a method of quantitatively determining a binding capacity of a surface adapted to contain reactive moieties.

Inventors discovered that a fluorescent moiety can be used to quantify a number of reactive moieties attached to a surface and consequently ascertain a number of biomolecules capable of binding to the surface via reactive moieties by measuring a signal emitted by the fluorescent moiety.

In the method of the invention, the fluorescent moiety is attached to the surface containing reactive moieties through a cleavable bond, preferably a disulfide bond or an aromatic azo group. The fluorescent moiety is preferably introduced by either a thiol-containing or thiol-reactive fluorophore.

The cleavable bonds are then cleaved to liberate the fluorescent moiety by methods known in the art, for example by using reducing agents.

Then, the liberated fluorescent moiety is detected using common instrumentation, such as a fluorimeter. Quantitative determination of the surface density of reactive moieties can be obtained by comparing the output signal for a surface on which the reactive moieties were not transformed into thiol or thiol-reactive groups (i.e., controls) to the output signal for a surface from which the fluorescent moiety was liberated.

A typical average difference in the concentrations of fluorophore, for example, dansyl-L-cysteine, between treated surfaces and the controls was about 0.4 μM, which corresponds to about 0.1 nmol/cm² of thiol-reactive groups on the surface. A linear correlation between the concentration of the fluorophore and the fluorescence intensity was found in the working range of 10⁻⁸-10⁻⁶ M. Calibration curves were made for each set of the fluorescence measurements.

The present invention also contemplates the use of fluorophores that comprise the requisite fluorescent moiety, a cleavable bond and a functional group. In this embodiment, the functional group reacts with the reactive moiety of the surface to form an uncleavable bond such that the cleaving predominantly occurs at the cleavable bond.

Suitable fluorophores comprise any fluorescent moiety and a cleavable bond between the fluorescent moiety and a functional group that can react with the surface reactive moieties. As described above, disulfide bonds represent one embodiment of a cleavable bond. In other instances, aromatic azo groups (i.e., R²—N═N—) serve as cleavable bond.

The method of the invention can be useful in a medical field, for example, for predicting and quantifying biologically active molecules to be bound or bound by reactive moieties of the surface.

The term “binding capacity,” as used in the present description, refers to the number of reactive moieties per unit area of the surface.

The term “biologically active molecules” as used in the present description, refers to, for example, proteins, cells, antibodies, and/or enzymes.

Surface

In the present invention, the surface is not limited to particular materials, but rather encompasses any material adapted to contain the requisite reactive moieties. Non-limiting examples of surfaces include metals, ceramics, biomaterials, semiconductors, and interpenetrating polymer networks. In one embodiment, the surface is polyurethane. Also, surfaces can be in a form of a film or a deposit on variety of materials such as, for example, metals, ceramics, biomaterials, semiconductors, and interpenetrating polymer networks.

The term “surface”, as used herein, denotes an interface (e.g., between phases, objects, ets.) comprised of more than one molecule.

Non-limiting example of biomaterials is a bioprosthetic tissue as disclosed in U.S. Pat. No. 5,674,298 to Levy et al., wherein the bioprosthetic tissue is stabilized with a polyphosphonate:polyepoxide monoadduct. The resulting reactive moiety attached to the bioprosthetic tissue is an epoxide, which can be used to determine the surface binding capacity of a bioprosthetic material utilizing the method of the invention.

Reactive Moiety

The surfaces can be modified with various reagents to obtain modified or derivatized surfaces containing reactive moieties. Preferably, the reactive moiety is a substututed thiol group or a thiol group itself.

In certain embodiments of the invention, reagents containing geminal bisphosphonate groups, which bind readily to, for example, a metal surface, are employed to anchor a variety of organic moieties to the metal surface. Preferably, such moieties comprise reactive moieties selected from the group consisting of a thiol, a substututed thiol, an epoxy and an amino group.

In certain embodiments of the invention, the reactive moieties undergo transformations before they are converted into the thiol or thiol-reactive groups. For example, where the surface comprises epoxy groups, the epoxy group can be ring-opened with a thiocarboxylic acid, such as, for example, thioacetic acid (MeC(O)SH), which contains a protected thiol group. Following deprotection, the surface would then present pendant thiol groups. Alternatively, an epoxy group can be ring-opened with a diamine, thereby furnishing a surface comprising amino groups.

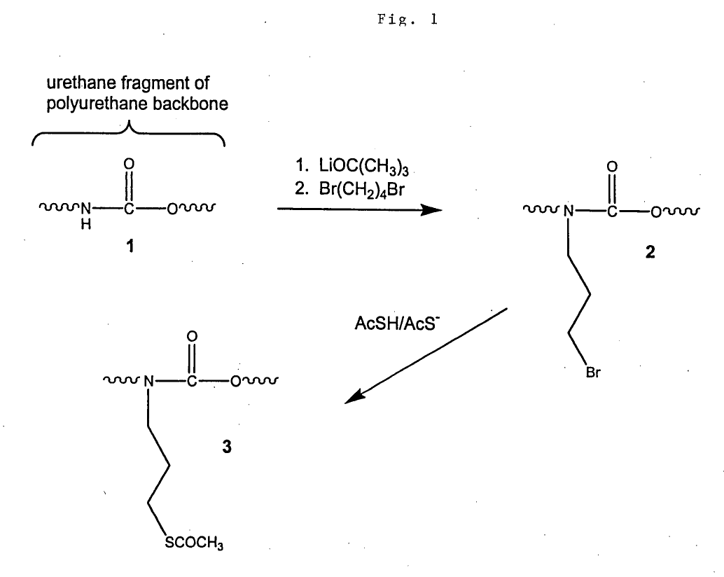

A non-limiting example of a surface containing reactive groups is polyurethane derivatized to contain protected thiol groups as shown in FIG. 1. A method of making the derivatized urethane is described in details in copending U.S. patent application entitled “NOVEL THIOL ACTIVATION OF POLYURETHANES AND METHODS OF MAKING THE SAME”, by Alferiev, Flshbein and Levy and copending U.S. patent application entitled “DERIVATIZED POLYURETHANE COMPOSITIONS WHICH EXHIBIT ENHANCED STABILITY IN BIOLOGICAL SYSTEMS AND METHODS OF MAKING THE SAME”, by Levy, Alferiev, and FIshbein filed on even date herewith.

Fluorophores

Thiol-Containing Fluorophores

In certain embodiments, the reactive moiety is a thiol group or amino group. Either of these groups can be reacted with a suitable reagent to furnish product 3 (as shown in FIG. 1) that comprises thiol-reactive groups. The preferred thiol-reactive group is a dithio group. Thus, for example, where the reactive moiety is a thiol group, treatment with a reactive dithio-containing reagent furnishes a surface with reactive dithio groups. An exemplary transformation in this context employs 5,5′-dithio-bis(2-nitrobenzoic acid) (“DTNB”):

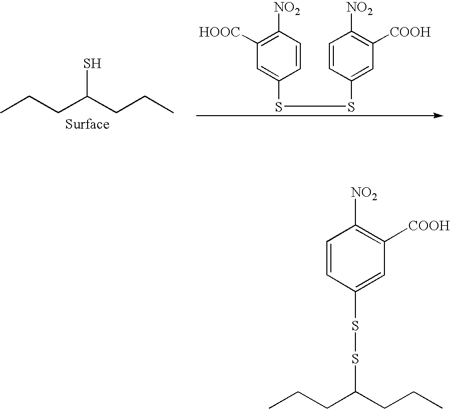

Similarly, surface amino groups can be transformed into reactive dithio groups of product 3 by using other dithio-containing reagents known to react with amino groups. Illustrative of this variant is the transformation depicted below, where succinimidyl 3-(2-pyridyldithio)propionate (“SPDP”) provides the dithio moiety:

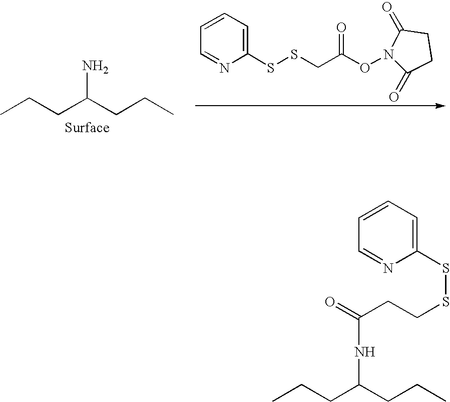

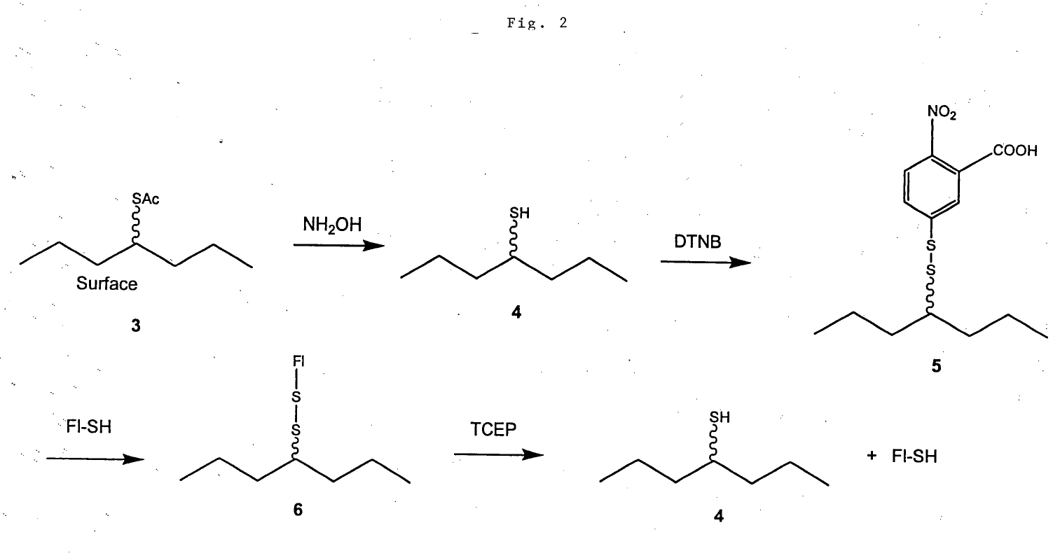

Next, thiol-reactive dithio groups of product 3 were contacted with a thiol-containing fluorophore, Fl—SH, whereby the fluorescent moiety (Fl), is tethered to the surface through a formation of a disulfide bond in product 6 as shown in FIG. 2. The invention contemplates a wide range of thiol-containing fluorophores, which can be realized by modifying any fluorescent moiety (Fl) with a thiol group. For example, reduction of compounds of the formula Fl—S—S—Fl is a useful way to prepare Fl—SH. A particularly preferred Fl—SH prepared in this manner is dansyl-L-cysteine as described in Example 5 below:

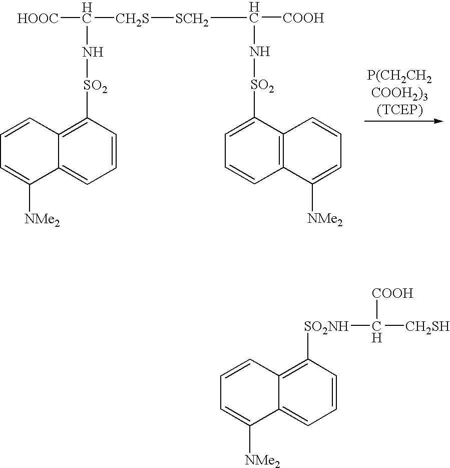

An analogous phosphine reduction of commercially available BODIPY-L-cystine (Molecular Probes) provides BODIPY-L-cysteine:

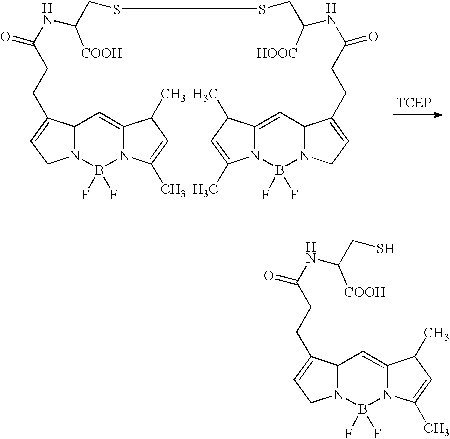

Other fluorescent moieties are derived from fluorescein derivatives. For example, mixtures of 5-[(2 or 3)-acetylmercaptosuccinoylamino]fluorescein (“SAMSA-fluorescein”) can be deprotected to provide the corresponding thiol-containing deprotected SAMSA-fluorescein as shown below:

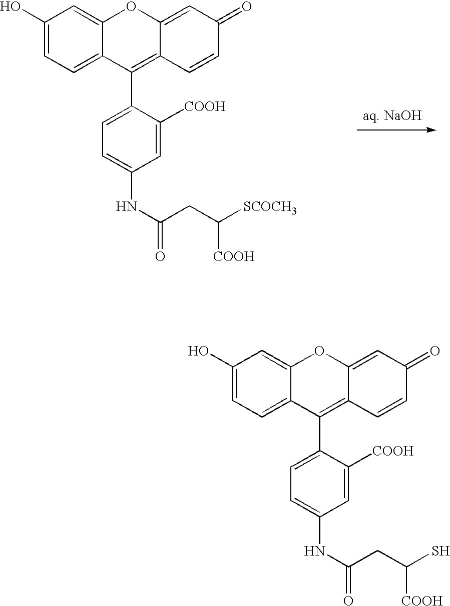

Contacting any of the thiol-containing reagents described above with the thiol-reactive groups in product 3 immobilizes the fluorescent moiety Fl on the surface via formation of disulfide bonds.

Thiol-Reactive Fluorophores

In certain embodiments of the invention, the reactive moieties are transformed into thiol groups of product 3. The transformation occurs by any well-known synthetic route directed to removal of a protective group. For example, polyurethane comprising pendant protected thiol groups, can be deprotected to generate a polyurethane comprising pending thiol groups.

Alternatively, surface amino groups can be transformed into surface thiol groups by using a variety of reagents. In one embodiment, such reagent is a Traut's reagent (2-iminothiolane):

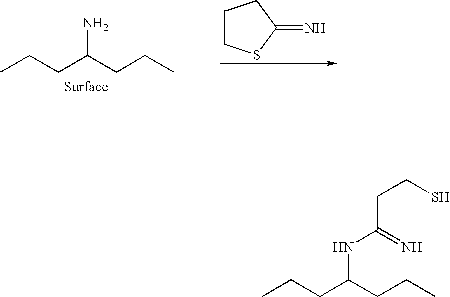

Surface epoxy groups can be ring-opened with a variety of reagents to present surface thiol groups. For example, an epoxy group can be treated with a diamine, followed by 2-iminothiolane as described above.

Next, thiol groups in product 3 are reacted with a thiol-reactive fluorophore, which results in the formation of disulfide bonds in product 6. The thiol-reactive fluorophore has a group capable of disulfide bond formation. Suitable thiol-reactive fluorophores include sulfenyl chlorides of general formula Fl—S—Cl and thiosulfonates of general formula Fl—S—SO₃(C₁₋₆ alkyl), each of which is capable of formally delivering a “Fl—S” moiety to surface thiol groups in product 3.

Particularly effective thiol-reactive fluorophores are fluorescent structures of a general formula Fl—S—S—R², where R² is a heterocyclic group or an electron-deficient group. Exemplary heterocyclic groups include pyridyl, preferably 2- or 4-pyridyl. An electron-deficient aromatic group is an aromatic hydrocarbon moiety, such as phenyl or naphthyl, which is substituted with one or more electron-withdrawing substituents. These include halo substituents such as fluoro, chloro, and bromo; nitro; nitrilo; carboxyl; esters; amides; and halogenated lower alkyl groups such as trifluoromethyl. A preferred heterocyclic group is pyridyl, such as is found in the effective thiol-reactive fluorophore N-[6-(7-amino-4-methylcoumarin-3-acetamido)hexyl]-3′-(2′-pyridyldithio)propionamide (“AMCA-HPDP”; Molecular Probes):

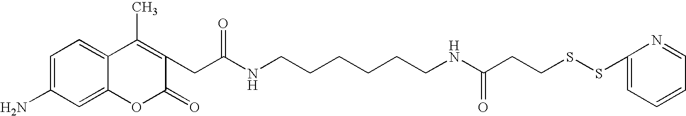

Other thiol-reactive fluorophores with pyridyldithio groups can be readily prepared from SPDP, described above, and any fluorescent compound having an amino group. An illustrative procedure in this context is the reaction between SPDP and tetramethylrhodamine cadaverine:

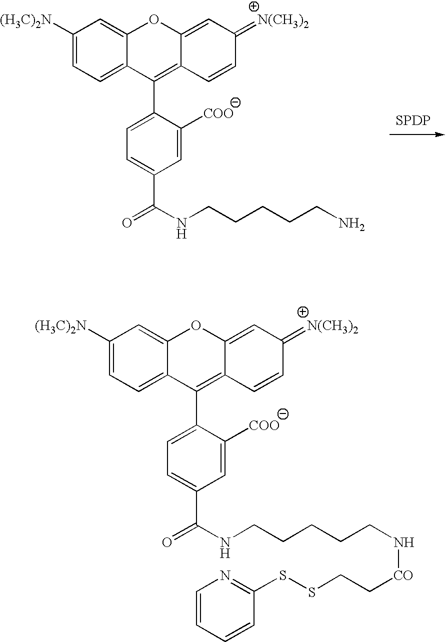

Fluorophores Comprising Cleavable Bridges

The present invention also contemplates the use of fluorophores that comprise cleavable bonds. In this context, the fluorophore reacts irreversibly with reactive moieties on the surface, and is then capable of liberating a detectable fluorescent compound through the cleavage of a bond, leaving a portion of the original fluorophore covalently bound to the surface.

Suitable fluorophores comprise any fluorescent moiety and a cleavable bond between the fluorescent moiety and a functional group that can react with the surface reactive moieties. As described above, disulfide bonds represent one embodiment of a cleavable bond. In other instances, aromatic azo groups (i.e., R²—NN—) serve as cleavable bonds. The fluorophore can be constructed from reagents containing the requisite fluorescent moiety, cleavable bond, and functional group. An illustrative procedure, depicted in the scheme below, commences with the coupling of commercially available dansyl-ethylenediamine with dithiobis-(3-propionic acid) in the presence of dicyclohexylcarbodiimide (“DCC”). The remaining carboxy group in the resultant product can then be activated with N-hydroxysuccinimide and N-ethyl-N′(3-dimethylaminopropyl)carbodiimide (“EDC”) to provide the N-succinimidyl ester functional group.

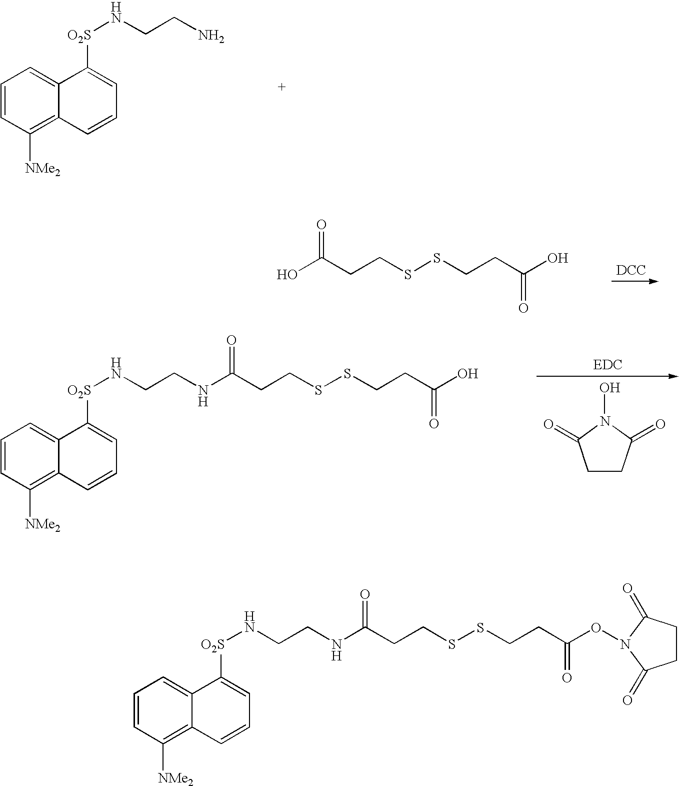

The functional group of the fluorophore described above is particularly reactive toward surface amino groups via the formation of non-cleavable amide bonds. Thus, although the fluorophore is immobilized on the surface, the fluorescent moiety can be liberated through the cleavage of the disulfide bond.

Measurement of Surface Binding Capacity

Once a fluorescent moiety, introduced by either a thiol-containing or thiol-reactive fluorophore, is attached to the surface through disulfide bonds, the surface is preferably washed to remove any unbound fluorophore. Suitable washes are those that preferably dissolve any unbound fluorophore and that cause no detachment of the covalently bound fluorophore. Exemplary washes include water, aqueous buffered solutions, and lower alcohols such as methanol and ethanol.

The disulfide bonds are then cleaved to liberate, for example, the fluorophore Fl—SH, as depicted schematically below. Reagents that are capable of cleaving disulfide bonds are well known in the art, and generally comprise reducing agents.

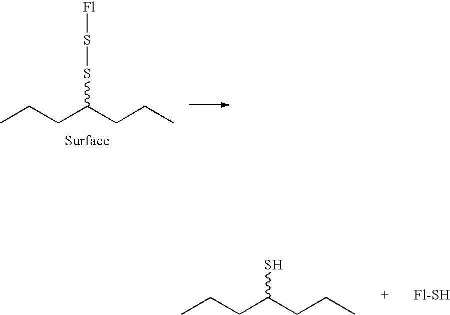

Suitable reducing agents include but are not limited to organic reagents such as dithiothreitol, β-mercaptoethanol, and mercaptoethylamine hydrochloride, and borohydrides such as sodium borohydride. Particularly preferred reducing agents are polar phosphines such as tris(2-cyanoethyl)phosphine and tris(2-carboxyethyl)phosphine (“TCEP”). Lower alkyl phosphines such as trimethylphosphine are also efficacious in this context.

In other embodiments comprising aromatic azo bonds, such azo bonds can be cleaved with aqueous salts of dithionite at elevated pH. An exemplary salt in this context is 0.1 M sodium dithionite, preferably at pH 9.

The liberated fluorophore then is detected using common instrumentation, such as a fluorimeter. Quantitative determination of the surface density of reactive moieties can be obtained by comparing the output signal for a surface on which the reactive moieties were not transformed into thiol or thiol-reactive groups (i.e., controls) to the output signal for a surface from which Fl—SH was liberated. Thus, a linear correlation between the concentration of liberated Fl—SH and fluorescence intensity yields the concentration of surface reactive moieties. In some cases, the inventors discovered that a substantial amount of thiol-containing or thiol-reactive fluorophore was absorbed irreversibly by the bulk of the surface. However, the use of controls effectively eliminates contributions from this phenomenon to the overall determination of surface binding capacity.

Materials and Apparatuses

Bruker Advance DMX 400 spectrometer was used for recording the NMR spectra reported herein. Medical grade polyether-urethane Tecothane TT1074A was obtained as pellets from Thermedics Inc. (Woburn, Mass.) and used without purification. Polymer, represented generally as 1 in FIG. 1, is based on 4,4′-methylenebis(phenyl isocyanate) (MDI), polytetramethylene ether glycol (PTMEG), and 1,4-butanediol as a chain extender. An analytical sample of the polyurethane was additionally purified by dissolution in dimethyl formamide (DMF), filtration, precipitation with a large volume of cold (−60° C.) methanol, washing with copious amounts of methanol then water, and vacuum-drying. ¹H NMR (DMF-d₇, the intensities are given in arbitrary units) δ 1.51-1. 76 (m, 1833H, CH₂CH₂ in the middle of tetramethylene bonds), 3.35-3.45 (m, 1620H, ether OCH₂), 3.87 (br. s, 105H, ArCH₂Ar), 4.10-4.17 (m, 211H, urethane OCH₂), 7.18 (m close to d, J=8 Hz, 215H, aromatic H, most likely in m-position to NH), 7.51 (br. m close to d, J=8 Hz, 212H, aromatic H, most likely in oposition to NH), 9.49 and 9.52 (two close br. s, total 100H, urethane NH). As calculated from the relative intensities of the aromatic protons and different types of CH₂ groups, the polyurethane contains 2.4 mmol/g of urethane groups.

### EXAMPLES

The invention will be illustrated in more detail with reference to the following Examples, but it should be understood that the present invention is not deemed to be limited thereto.

### Example 1

### Preparing Polyurethane With Pendant 4-Bromobutyl Substituents

This example demonstrates a method of derivatizing polyurethane by using a multifunctional linking reagent.

The polyurethane as described above (15.8 g, containing ca. 38 mmol of urethane NH groups) was soaked in toluene (150 ml) for 60 hours. After removal of the excess solvent, the swollen polymer was dried in vacuo at 40° C. and dissolved in dry N,N-dimethylacetamide (DMAc) (300 ml) under a flow of dry argon.

Freshly distilled 1,4-dibromobutane (15 ml, 126 mmol) was added, the solution was cooled to −6° C., and a 1.0 M solution of lithium tert-butoxide in hexanes (Sigma-Aldrich, 7.6 ml, 7.6 mmol) diluted with dry DMAc (20 ml) was added over a 10-minute period with vigorous stirring at −5 to −6° C. The resultant mixture was stirred at −1° to 1° C. for 1 hour with continued argon protection and then acidified with acetic acid (6.5 ml).

The reaction solution was poured into a large volume (1200 ml) of cold (−55° C.) methanol, the resulting coagulate of polymer was separated, thoroughly washed with methanol followed by 2-propanol, and dried in vacuo (0.5 mm Hg) at room temperature.

The crude polymer was redissolved in DMF (275 ml), the solution was filtered, and the polymer was precipitated with cold methanol, washed with large volumes of methanol and water, stirred for 16 hours with a large amount of water at 4° C. and dried in vacuo (0.04 mm Hg) at room temperature to yield 15.64 g of the polyurethane derivative represented generally as 2 in FIG. 1. ¹H NMR spectral analysis of 2 showed that the concentration of bromobutyl groups was 0.45 mmol per gram of 2.

### Example 2

### Preparation of a Polyurethane Having Pendant Acetylthiobutyl Substituents

This example demonstrates the preparation and thermal stability of a polyurethane having pendant protected thiol groups.

Polyurethane 2 (15.5 g, containing ca. 7.1 mmol of pendant bromobutyl groups) as prepared in Example 1 was dissolved in dry DMAc (220 ml) under a flow of argon, and the solution was cooled to −8° C. Freshly vacuum-distilled (at 115 mm Hg) thiolacetic acid (5.72 ml, 80 mmol), together with a freshly prepared 0.25M DMAc-solution of tetrabutylammonium tetraborate (Bu₄N)₂B₄O₇ (80 ml, 20 mmol), was introduced. The temperature was not allowed to exceed 0° C.

The mixture was stirred at −1° to 1° C. for 1 hour with continued Ar protection and then poured into a large volume (1400 ml) of cold methanol (−60° C.). The resulting coagulate of polymer was separated, washed and dried as described in Example 1.

The crude polymer was redissolved in DMF (300 ml), filtered, precipitated with cold methanol, washed with large volumes of methanol and water, stirred for 4 hours with a large amount of water at room temperature and dried at 0.04 mm Hg to yield 14.43 g of the polyurethane represented generally as 3 in FIG. 1. ¹H NMR spectral analysis of 3 (FIG. 1) showed that the polyurethane contained 0.45 mmol of acetylthio groups per gram of polyurethane and that it contained no unreacted bromobutyl groups.

The acetylthio-modified polyurethane 3 is similar to starting polyurethane 1 in both visual appearance and propensity for water absorption. A sample of polyurethane 3 was heated in vacuo at 209°-214° C., which is the highest temperature recommended by the manufacturer for the thermoprocessing of polyurethane 1. After 5 minutes, polyurethane 3 exhibited no visual changes and no spectral changes (as determined by ¹H NMR) relative to a sample of polyurethane 3 that was not heated.

### Example 3

### Formation of Films Prepared from Polyurethane having Pendant Acetylthiobutyl Substituents

This example demonstrates the preparation of surfaces in the form of films of the derivatized polyurethane.

Films of the polyurethane described in Example 2 were cast on a Teflon-coated surface using ca. 6% filtered solutions in freshly distilled THF (free of peroxides) in air at room temperature. The cast films were dried in a flow of air for 2-3 days, thoroughly washed with water, and then air-dried. The films exhibited an average thickness of about 0.2 mm.

### Example 4

### Preparation of Polyurethane having Pendant Butylthiol Substituents

This example demonstrates the deprotection of protected thiol groups to obtain a polyurethane having pendant thiol groups.

The polyurethane films of Example 3 were cut into rectangles (1.3×0.8 cm, total surface area ca. 2 cm²). The films were soaked for 1.5 h in a deoxygenated aqueous solution of hydroxylamine hydrochloride (0.6M), NaOH (0.51M), ethylenediaminetetraacetic acid (EDTA; free acid, 0.3 mg/ml), Na₂HPO₄ (52 mM) and sodium dodecyl sulfate (0.1 mg/ml) at 20°-22° C. under a blanket of Ar.

The films were removed from the hydroxylamine solution and rinsed briefly with a 2 mM solution of EDTA disodium salt. The resulant polyurethane, represented generally as 4 in FIG. 2, has pendant thiol groups, the concentration of which was determined in the assay method exemplified below.

### Example 5

### Preparation of Dansyl-L-Cysteine

This example demonstrates the synthesis of a thiol-reactive fluorophore.

Didansyl-L-cystine (Sigma-Aldrich, 95% pure, 201 mg, 0.28 mmol) was dissolved in methanol (5.4 ml) under Ar, and treated with a solution of tris(2-carboxyethyl)phosphine (TCEP) hydrochloride (Pierce, 107 mg, 0.37 mmol) and NaHCO3 (81 mg, 0.96 mmol) in water (1.1 ml). The resultant mixture was stirred at 20-22° C. for 5 minutes, diluted with 2-propanol (10 ml), and the solvents were quickly removed in vacuo at 20°-25° C. It was found that longer reaction times and delays in the removal of solvents caused formation of a non-separable by-product.

The residue was extracted with CHCl₃ (total ca. 15 ml), and the solution was filtered through a layer of cellulose powder. After removal of CHCl₃, the residue (ca. 250 mg) was purified by flash-chromatography on a small amount of silica gel in CHCl₃—MeOH (100:0 then 95:5 by volume). It was important to finish the chromatography in less than 0.5 hour. Otherwise, a significant loss of dansyl-L-cysteine occurs, most likely due to its oxidation promoted by silica gel.

The purified product, which was a syrup after the removal of solvents, was dissolved in EtOAc (2.5 ml), diluted with n-heptane (4 ml) and slowly dried in vacuo at 20°-23° C., resulting in the solidification of the product. The residual solid powder was further dried at 0.1 mm Hg for 1 hour to yield 152 mg (75%). Dansyl-L-cysteine was characterized by TLC (silica gel, CHCl3—MeOH—AcOH, 95:5:2): Rf ca. 0.4; and ¹H NMR (CDCl3) δ 1.36 (br., 1H, SH), 2.69 and 2.79 (two br. d, J=14 Hz, 1H and 1H, diastereotopic CH₂), 2.91 (s, 6H, CH₃), 4.17 (m, 1H, CH), 5.89 (br. d, J=7 Hz, 1H, NH), 7.24 (d, J=7 Hz, 1H, Ar—H), 7.50 (dd, J=8, 7 Hz, 1H, Ar—H), 7.60 (m close to t, J=8 Hz, 1H, Ar—H), 8.24 (dd, J=7, 1 Hz, 1H, Ar—H), 8.35 (d, J=9 Hz, 1H, Ar—H), 8.50 (d, J=9 Hz, Ar—H).

### Example 6

### Determination of Surface Binding Capacity of Polyurethane having Pendant Butylthiol Substituents

This example demonstrates the quantification of thiol groups that are presented by a surface of a polyurethane containing pendant thiol substituents.

The polyurethane films of Example 4 were treated for 1 hour at 20°-22° C. with a solution (pH=7) of with 5,5′-dithiobis(2-nitrobenzoic acid) (DTNB; Sigma-Aldrich, 149 mg), KHCO₃ (68 mg), water (1.6 ml) and K₂HPO₄ (0.18 mmol) to provide a polyurethane with pendant disulfide substituents (represented generally as 5 in FIG. 2). Untreated films (i.e., those of Example 4) served as controls.

After washing with 0.1M phosphate buffer (pH=7), the films were exposed to a solution of dansyl-L-cysteine (21 mg) of Example 5 (represented generally as Fl—SH in FIG. 2), Na₂HPO₄ (0.05 mmol) and NaH₂PO₄ (0.16 mmol) in water (10 ml) for 20 minutes to provide a polyurethane with pendant disulfide substituents (represented generally as 6 in FIG. 2). To remove the unbound dansyl-L-cysteine, the films were stirred with large volumes of a mixture containing Na₂HPO₄ (6 mM), NaH₂PO₄ (6 mM), sodium dodecyl sulfate (0.2 mg/ml) and N-ethylmaleimide (Sigma-Aldrich, 0.24 mg/ml) for 5 days at 4° C.

Finally, the films were individually incubated with stirring in 0.5 ml of methanolic solution containing TCEP hydrochloride (5 mg/ml) and NaOAc (2.9 mg/ml) for 20 minutes at 20°-22° C. to furnish polyurethane 3 and liberate dansyl-L-cysteine (Fl—SH; FIG. 2). The concentrations of released dansyl-L-cysteine (Fl—SH) were determined using a Victor² fluorometer, model 1420 (Wallac, Finland) with a set of filters providing excitation at 355 nm and emission at 535 nm.

A typical average difference in the concentrations of dansyl-L-cysteine between the DTNB-treated films and the controls was 0.4 μM, which corresponds to 0.1 nmol/cm² of thiol-reactive groups on the surface of the polyurethane films. A linear correlation between the concentration of dansyl-L-cysteine and the fluorescence intensity was found in the working range of 10⁻⁸-10⁻⁶ M. Calibration curves were made for each set of the fluorescence measurements.

While the invention has been described in detail and with reference to specific examples thereof, it will be apparent to one skilled in the art that various changes and modifications can be made therein without departing from the spirit and scope thereof.

What is claimed is:

1. A method of determining a binding capacity of a surface, the method comprising:
providing the surface containing a reactive moiety;
providing a fluorophore comprising a fluorescent moiety adapted to emit a detectable signal;
reacting the fluorophore with the reactive moiety to form a linking bond between the fluorophore and the reactive moiety;
cleaving a cleavable bond to liberate the fluorescent moiety; and
detecting the detectable signal to determine the binding capacity of the surface.

2. A method of determining a binding capacity of a surface, the method comprising:
providing the surface containing a reactive moiety;
providing a fluorophore comprising a fluorescent moiety adapted to emit a detectable signal;
reacting the fluorophore with the reactive moiety to form a linking bond between the fluorophore and the reactive moiety, wherein the linking bond is the cleavable bond and is a disulfide bond or an aromatic azo group;
cleaving a cleavable bond to liberate the fluorescent moiety; and
detecting the detectable signal to determine the binding capacity of the surface.

3. The method of claim 2, wherein the cleavable bond is a disulfide bond.

4. The method of claim 2, wherein the aromatic azo group is represented by a formula:
—R²—N═N—
wherein R² is an aromatic compound selected from the group consisting of a heterocyclic group and an electron-deficient aromatic group.

5. The method of claim 2, wherein the fluorophore is a thiol-containing fluorescent structure represented by a formula:
Fl—SH
wherein Fl is the fluorescent moiety and is a member selected from the group consisting of fluorescent L-cysteine, BODIPY-L-cysteine, fluorescein and derivatives thereof.

6. The method of claim 5, wherein the thiol-containing fluorescent structure is a member selected from the group consisting of:

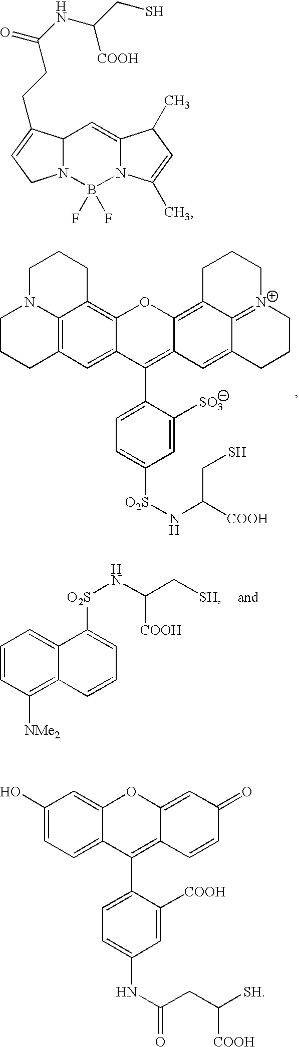

7. The method of claim 5, wherein the thiol-containing fluorescent structure is

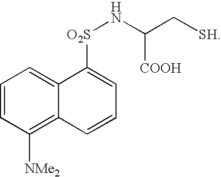

8. The method of claim 5, wherein the thiol-containing fluorescent structure is:

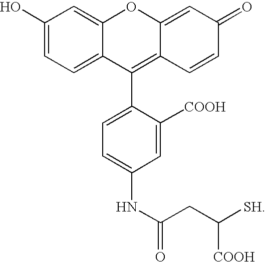

9. The method of claim 2, wherein the fluorophore is a thiol-reactive fluorescent structure represented by a formula:
Fl—S—X
wherein X is a member selected from the group consisting of Cl, SO₃(C₁-C₆ alkyl), and S—R², wherein R² is a heterocyclic group or an electron-deficient aromatic group.

10. The method of claim 9, wherein R is a pyridyl group or a phenyl group substituted with one or more electron-withdrawing substituents.

11. The method of claim 9, wherein the thiol-reactive fluorescent structure is a member selected from the group consisting of:

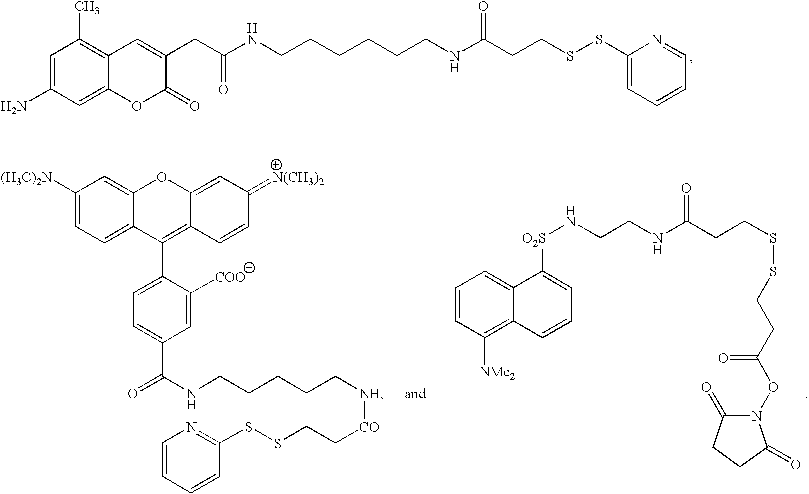

12. The method of claim 2, wherein the fluorophore further comprises a functional group, wherein the functional group is bound to the fluorescent moiety by the cleavable bond and is reacted with the reactive moiety to form an uncleavable bond such that cleaving predominantly occurs at the cleavable bond.

13. The method of claim 12, wherein the functional group is a member selected from the group consisting of an amino group, a thiol group, a protected thiol group, and an epoxy group.

14. The method of claim 2, wherein the surface is a member selected from the group consisting of a polymer, a metal, a biomaterial, a ceramic, and a semiconductor.

15. The method of claim 14, wherein the polymer is polyurethane.

16. The method of claim 2, wherein the reactive moiety is a thiol, a thiol-reactive group or a group adapted to be converted into a thiol or a thiol-reactive group.

17. The method of claim 2, wherein the reactive moiety is a thiol group or an amino group.

18. The method of claim 17, wherein the reactive moiety is further reacted with 5,5′-dithio-bis(2-nitrobenzoic acid) or succinimidyl 3-(2-pyridyldithio)propionate.

19. The method of claim 2, wherein the reactive moiety is a dithio group.

20. The method of claim 2, wherein the cleavable bond is cleaved by using a reducing agent selected from the group consisting of dithiothreitol, β-mercaptoethanol, mercaptoethylamine hydrochloride, a borohydride, and a phosphine.

21. The method of claim 20, wherein the borohydride is sodium borohydride.

22. The method of claim 20, wherein the phosphine is a member selected from the group consisting of tris(2-cyanoethyl)phosphine, tris(2-carboxyethyl)phosphine and trimethylphosphine.

23. A kit for practicing of method of claim 2, the kit comprising a fluorophore.

24. The kit of claim 23, wherein the fluorophore comprises the fluorescent moiety and a linking bond precursor.

25. The kit of claim 23, wherein the linking bond precursor is adapted to form a cleavable disulfide bond or an aromatic azo group.

26. The kit of claim 25, wherein the linking bond precursor is —SH.

27. The kit of claim 25, wherein the linking bond precursor is represented by a formula:
—S—X
wherein X is a member selected from the group consisting of Cl, SO₃(C₁-C₆ alkyl), and S—R², wherein R² is a heterocyclic group or an electron-deficient aromatic group . . .

28. The kit of claim 23, wherein the fluorophore further comprises a functional group, wherein the functional group is bound to the fluorescent moiety by the cleavable bond and is adapted to react with the reactive moiety to form an uncleavable bond.

29. The kit of claim 28, wherein the functional group is a member selected from the group consisting of an amino group, a thiol group, a protected thiol group, and an epoxy group.

30. The kit of claim 28, wherein the uncleavable bond is an amide bond.
